(19) United States
(12) Patent Application Publication
(10) Pub. No.: US 2018/0221362 A1
(43) Pub. Date: Aug. 9, 2018

(54) EZH2 INHIBITORS AND MODULATION OF REGULATORY T-CELL FUNCTION

(71) Applicant: Constellation Pharmaceuticals, Inc., Cambridge, MA (US)

(72) Inventors: Srimoyee Ghosh, Lexington, MA (US); Jose M. Lora, Boston, MA (US)

(21) Appl. No.: 15/749,890

(22) PCT Filed: Jul. 28, 2016
(86) PCT No.: PCT/US2016/044409

§ 371 Date: Feb. 2, 2018

Related U.S. Application Data
(60) Provisional application No. 62/200,244, filed on Aug. 3, 2015.

(51) Int. Cl. A61K 31/4545; A61P 35/00; C07K 16/28; A61K 39/395

(52) U.S. Cl. CPC A61K 31/4545; A61P 35/00; A61K 2039/505; A61K 39/3955; C07K 16/2818

(57) ABSTRACT

Provided herein are methods of treating cancers characterized by a high frequency of one or more suppressive immune cells, comprising administering a therapeutically effective amount of an enhancer of zeste homolog 2 (EZH2) inhibitor. Also provided are combination therapies using an EZH2 inhibitor and a second agent that is an immunomodulator.

### RELATED APPLICATIONS

This application claims priority to U.S. provisional application No. 62/200,244, filed Aug. 3, 2015, the contents of which are incorporated herein in their entirety.

### BACKGROUND

A number of small molecule inhibitors of Enhancer of Zeste Homolog 2 (EZH2) are in clinical development for the treatment of various types of cancer. Despite advances toward the development of new EZH2 inhibitors as anti-cancer therapies, patient populations for which these inhibitors are particularly effective remains unknown. Disclosed herein are patient populations that are particularly suited for treatment with an EZH2 inhibitor.

### SUMMARY

It has now been found that inhibition of EZH2 reduces the proliferation of regulatory T-cells (Tregs), increases cytotoxic T-cells (CD8), produces favorable CD8/Treg ratios, increases natural killer (NK) and natural killer T-cells (NKT), and reduces M2 tumor-associated macrophages (TAMs). Based on these discoveries subjects having cancers characterized by a high frequency of Tregs or a high frequency of M2 associated macrophages, or subjects in need of immune response to a cancer, represent a viable patient population for effective anti-cancer treatment using an EZH2 inhibitor.

For example, treatment with EZH2 inhibitors was found to reduce the suppressive capacity of human regulatory T cells in vitro (see FIG. 4). In vivo, EZH2 inhibition was found to reduce proliferation of regulatory T-cells (Tregs), increase cytotoxic T-cells (CD8), produce favorable CD8/Treg ratios, increase natural killer (NK) and natural killer T-cells (NKT), and reduce M2 tumor-associated macrophages (TAMs). See e.g., FIGS. 6-8. In addition, while CT26 carcinoma cells were found to be insenstite to EZH2 inhibition in vitro (FIG. 9), treatment of mice (i.e., in vivo) with an EZH2 inhibitor reduced CT26 carcinoma cell growth. See e.g., FIG. 10 and FIG. 14.

Tregs are abundant in tumors and are a major component in cancer progression as they have a crucial role in cancer promotion via suppression of anti-cancer immune responses and even nonimmune-mediated mechanisms. See e.g., Farashi-bonab et al., MOJ Immunol 2014, 1(4): 00024. Modulation of Treg-inducing factors in the tumor microenvironment and depletion or blocking of Tregs have been shown to be valuable approaches for induction of anti-cancer immunity and improving the efficacy of immunotherapies, especially in contexts in which increased levels of Tregs in tumors are detected and associated with poor disease outcome. See e.g., Nizar et al., British Journal of Cancer (2009) 100, 1697-1703. Even a single manipulation that depletes Tregs in tumors has been shown to serve as effective cancer monotherapy as well as serve as an approach that can be combined well with other cancer therapies. See e.g., Smyth et al., Immunology and Cell Biology (2014) 92, 473-474.

Because EZH2 inhibitors are shown herein to effectively reduce proliferation of regulatory T-cells (Tregs, see e.g., FIG. 6) and given the known connection between Treg suppression and anti-cancer immunotherapies, in one aspect, provided herein are methods for treating a subject with a cancer having a high frequency Tregs, comprising administering an effective amount of an EZH2 inhibitor. Such methods further comprise administering a therapeutically effective amount of a second agent that is an immunomodulator.

Cytotoxic T-cells (also known as CD8+ T-cells or killer T-cells) are T-lymphocytes that kill cancer cells, cells that are infected, or cells that are damaged or infected in other ways. See e.g., Maher et al., British Journal of Cancer (2004) 91, 817-821. Based on this data, and because EZH2 inhibitors are shown herein to increase cytotoxic T-cells (see FIG. 6), in another aspect, provided herein are methods of increasing the frequency of cytotoxic T-cells in a subject having cancer, comprising administering to the subject an effective amount of an EZH2 inhibitor. Such methods further comprise administering a therapeutically effective amount of a second agent that is an immunomodulator.

NK cells are known to play a role in tumor immunosurveillance by e.g., directly inducing the death of tumor cells. See e.g., Zamai et al., J Immunol 2007; 178:4011-4016. NKT cells share properties with NK cells and can coexpress semi-invariant T-cell receptor and NK cell markers. See e.g., Godfrey Nat. Rev. Immunol. 4 (3): 231-7. Based on the connections between NK cells and NKT cells in cancer treatment, because EZH2 inhibitors are shown herein to increase NK and NKT cells (see e.g., FIG. 7), in another aspect, provided herein are methods of increasing the frequency of NK cells or NKT cells, or both, in a subject having cancer, comprising administering to the subject an effective amount of an EZH2 inhibitor. Such methods may further comprise administering a therapeutically effective amount of an immunomodulator.

Tumor-associated macrophages (TAMs) are classified into two major phenotypes, M1 and M2. M1 TAMs suppress cancer progression, while M2 TAMs promote tumor growth. See e.g., Zhang et al. Journal of Ovarian Research 2014, 7:19 and Heusinkveld Journal of Translational Medicine 2011, 9:216. Based on this data, because EZH2 inhibitors are shown herein to reduce M2 tumor-associate macrophages (see e.g., FIG. 8), in another aspect, provided herein are methods of treating a subject with a cancer characterized by a high frequency of M2 TAMs, comprising administering to the subject a therapeutically effective amount of an EZH2 inhibitor. Such methods further comprise administering a therapeutically effective amount of a second agent that is an immunomodulator.

Also provided herein are pharmaceutical compositions comprising an EZH2 inhibitor and a second agent that is an immunomodulator. It has been found that this combination produces a synergistic effect in reducing the proliferation of regulatory T-cells (Tregs), increasing cytotoxic T-cells (CD8), producing favorable CD8/Treg ratios, increasing natural killer (NK) and natural killer T-cells (NKT), and reducing M2 tumor-associate macrophages (TAMs). See e.g., FIGS. 11-13. Synergism was also seen upon administration in vivo, where the combination was found to reduce carcinoma cells. See e.g., FIG. 10 and FIG. 14.

### BRIEF DESCRIPTION OF THE FIGURES

FIG. 1 illustrates PRC2 core components up-regulated during human Treg differentiation.

FIG. 2 illustrates that EZH2 binds to repressed loci in Treg cells, and inhibition results in loss of H3K27 tri-methylation.

FIG. 3A illustrates that EZH2 inhibition had no impact in FOXP3 expression, FIG. 3B illustrates a descrease in H3K27me3 levels in treated FOXP3⁺ T-cells, and FIG. 3C illustrates a dose-dependent increase in the expression of certain cytokine levels.

FIG. 4 illustrates that EZH2 catalytic activity is required for suppressive capacity of Treg cells.

FIG. 5 illustrates that EZH2 knockdown in human iTregs impairs suppressive function.

FIG. 6 illustrates a reduction in the proliferation of regulatory T-cells (Tregs) and an increase in the proliferation of cytotoxic CD8 T cells upon treatment with an EZH2 inhibitor.

FIG. 7 illustrates an increase in NK cells upon treatment with an EZH2 inhibitor.

FIG. 8 illustrates a reduction in suppressive M2 TAMs upon treatment with an EZH2 inhibitor.

FIG. 9 illustrates an in vitro experiment on the sensitivity of CT26 cells upon treatment with cisplatin and an EZH2 inhibitor.

FIG. 10 illustrates an in vivo reduction in CT26 tumor volume upon treatment with an EZH2 inhibitor and upon treatment with an EZH2 inhibitor and a second agent that is an immunomodulator.

FIG. 11 illustrates a reduction in the proliferation of regulatory T-cells (Tregs) and an increase in the proliferation of cytotoxic CD8 T cells upon treatment with an EZH2 inhibitor and a second agent that is an immunomodulator.

FIG. 12 illustrates and increase in NK and NKT cells upon treatment with a an EZH2 inhibitor and a second agent that is an immunomodulator.

FIG. 13 illustrates a reduction in suppressive M2 TAMs upon treatment with an EZH2 inhibitor and a second agent that is an immunomodulator.

FIG. 14A illustrates that CT26 carcinoma cells are not sensitive to EZH2 inhibition in vitro, whereas FIG. 14B illustrates an in vivo reduction in CT26 tumor volume upon treatment with an EZH2 inhibitor and upon treatment with an EZH2 inhibitor and a second agent that is an immunomodulator.

### DETAILED DESCRIPTION

It has been found that the adminstraton of an EZH2 inhibitor elicts an immune response by one or more of the markers described herein. Based on this discovery, the present disclosure is directed to, in one aspect, methods for treating a particular population of cancer subjects using an EZH2 inhbitor. This population of cancer subject's comprises cancers that are charatercized as having a high frequency of one or more suppressive immune cells.

In one aspect, the present disclosure provides a method of treating a subject with a cancer characterized by a high frequency of one or more suppressive immune cells, comprising administering to the subject a therapeutically effective amount of an EZH2 inhibitor.

In one aspect, prior to treatment with a therapeutically effective amount of an EZH2 inhibitor, the cancer was determined to comprise a high frequency of one or more suppressive immune cells. Such a determination can be made by routine diagnostics methods. These methods include, but are not limited to, biopsy, endoscopy, diagnostic imaging (X-ray, CAT scan, MRI, and ultrasound), and blood tests. In one aspect, the step of performing a biopsy of the subject's cancer prior to treatment and determining if the cancer comprises a high frequency of one or more suppressive immune cells is performed prior to treatment with a therapeutically effective amount of an EZH2 inhibitor.

In another aspect, provided herein is a method of treating a subject with a cancer comprising determining the frequency of one or more suppressive immune cells in the cancer; and administering to the subject a therapeutically effective amount of an EZH2 inhibitor, if the subject's cancer comprises a high frequency of one or more suppressive immune cells.

In another aspect, provided herein is a method of assessing the efficacy of an EZH2 inhibitor to treat cancer in patient comprising obtaining a sample from the patient and determining the frequency of one or more suppressive immune cells of the cancer, wherein the EZH2 inhibitor is likely to be effective if the frequency of one or more suppressive immune cells is high.

In another aspect, provided herein is a method of treating a subject with a cancer, comprising determining the frequency of one or more suppressive immune cells of the cancer and administering to the subject a therapeutically effective amount of a cancer therapy other than the administration of an EZH2 inhibitor, if the frequency of one or more suppressive immune cells of the subject's cancer is not high; and administering a therapeutically effective amount of an EZH2 inhibitor, if the frequency of one or more suppressive immune cells of the subject's cancer is high.

In another aspect, the methods described herein further comprise administering a therapeutically effective amount of an immunomodulator. It will be understood that unless otherwise indicated, the administrations described herein include administering a described EZH2 inhibitor prior to, concurrently with, or after administration of an immunomodulator described herein. Thus, simultaneous administration is not necessary for therapeutic purposes. In one aspect, however, the EZH2 inhibitor is administered concurrently with the immunomodulator.

As defined herein, “immunomodulator” refers to an agent that is responsible for inducing or enhancing an immune response to a cancer in a patient such that the patient's immune system is able to slow the progression, retard, reduce the patient's cancer or reduce the spread of cancer. Such agents include e.g., immune checkpoint blockade inhibitors, cell based therapies, vaccination strategies, agents that prevent metabolic inhibition of immune responses, and cytokine-based therapies. In one aspect, the immunomodulator of the present methods is an immune checkpoint blockade inhibitor. In one aspect, the immunomodulator described herein is an immune checkpoint blockade inhibitor selected from anti-CTLA4, ipilimumab, nivolumab, pembrolizumab, pidilizumab, BMS 936559, atezolizumab, anti-CD47, PD-1 antibody, anti-PDL1, lambrolizumab, AMP-224, and MEDI-4736. In one aspect, the immunomodulator described herein is an immune checkpoint blockade inhibitor selected from anti-CTLA4, ipilimumab, nivolumab, pembrolizumab, pidilizumab, BMS 936559, atezolizumab, anti-CD47, PD-1 antibody, anti-PDL1, avelumab, lambrolizumab, AMP-224, and MEDI-4736. In another alternative, the immunomodulator described herein is aPD-1 antibody.

As defined herein “suppressive immune cells” refer to those of the lymphoid lineage (e.g., T lymphocytes, B lymphocytes, and natural killer cells) and those of the myeloid lineage (e.g., monocytes, macrophages, langerhans cells, dendritic cells, megakaryocytes, and granulocytes (eosinophils, neutrophils, basophils) that can suppress the activity or proliferation of other immune cells included in a patient's defenses against cancer. In one aspect, the one or more suppressive immune cells as recited in the methods described herein are selected from regulatory T-cells (Tregs), cytotoxic T-cells (CD8), natural killer (NK) and natural killer T-cells (NKT), and M2 tumor-associate macrophages (TAMs), and combinations thereof. In another aspect, the one or more suppressive immune cells as recited in the methods described herein are regulatory T cell or M2 tumor associated macrophages, or a combination thereof.

Also provided herein are methods of treating a subject with a cancer comprising administering a therapeutically effective amount of an EZH2 inhibitor; determining if after administration of the EZH2 inhibitor a reduction in Treg-mediated suppression of T cell proliferation occurred, a suppression of M2 tumor-associated macrophages occurred, or an increase in the frequency of natural killer cells (NK) cells occurred, or a combination thereof; and continuing to administer a therapeutically effective amount of an EZH2 inhibitor if there has been a reduction in Treg-mediated suppression of T cell proliferation, a suppression of tumor-associated macrophages, or an increase in the frequency of natural killer cells (NK) cells, or a combination thereof. If not, the subject is treated with an anticancer therapy different than EZH2.

Further provided are methods of treating a cancer in a subject comprising taking a sample of the cancer and determining if there is a high frequency of one or more high frequency of regulatory T cells or a high frequency of M2 tumor associated macrophages and treating the subject with an EZH2 ihibitor. If the subject does not have a high frequency of one or more high frequency of regulatory T cells or a high frequency of M2 tumor associated macrophage, then one may treat the subject with an anticancer therapy other than an EZH2 inhibitor.

Thus, provided herein are methods of treating a subject with a cancer comprising administering a therapeutically effective amount of an EZH2 inhibitor; determining if after administration of the EZH2 inhibitor a reduction in Treg-mediated suppression of T cell proliferation occurred, a suppression of M2 tumor-associated macrophages occurred, or an increase in the frequency of natural killer cells (NK) cells occurred, or a combination thereof; and administering to the subject a therapeutically effective amount of a cancer therapy other than the administration of an EZH2 inhibitor if a reduction in Treg-mediated suppression of T cell proliferation did not occur, a suppression of tumor-associated macrophages did not occur, and an increase in the frequency of natural killer cells (NK) cells did not occur; and continuing to administer a therapeutically effective amount of an EZH2 inhibitor if there has been a reduction in Treg-mediated suppression of T cell proliferation, a suppression of M2 tumor-associated macrophages, or an increase in the frequency of natural killer cells (NK) cells, or a combination thereof.

As used herein “high frequency” means a median cut-off value of 1.5, 2, 2.5, 3, 3.5, 4, 4.5, 5, 5.5, 6, or 6.5 intratumor Tregs or M2 tumor associated macrophages in a tissue sample taken from a cancer per high-powered microscopic field (400× magnification). In one aspect, the median cut-off value is 1.5, 2, 2.5, or 3 intratumor Tregs or M2 tumor associated macrophages in a tissue sample taken from a cancer per high-powered microscopic field (400× magnification). In another aspect, the median cut-off value is 2 intratumor Tregs or M2 tumor associated macrophages in a tissue sample taken from a cancer per high-powered microscopic field (400× magnification). Methods for determining intratumor Tregs or M2 tumor associated macrophages per high-powered microscopic field (400× magnification) are known in the art and can be found in e.g., Gao et al (2007) J. Clin Onc 25(18): 2586-2593.

In addition to the above, high frequency of M2 tumor associated macrophages also means a density of 20, 25, 30, 35, 40, 45, 50, 55, or 60 M2 macrophages for approximately 20,000 total cells per tumor sample. In another aspect, the mean density is 20, 25, or 30 M2 macrophages for approximately 20,000 total cells per tumor sample. Methods for determining the density of M2 macrophages per total cells in a tumor sample are known in the art and include e.g., performing laser capture microdissection (LCM)-based flow cytometry on immunostained 15-μm sections of paraffin-embedded cancer specimens. See e.g., Zhang et al. Journal of Ovarian Research 2014, 7:19.

As used herein, “increase the frequency” of one or more of the cytotoxic immune cells defined herein, such as in e.g., an increase in the frequency of natural killer cells, means increasing the activity, proliferation, or development of one or more of the cytotoxic immune cells in a patient after treatment relative to prior to treatment.

As used herein “a reduction in” one or more of the suppressive immune cells defined herein, such as in e.g., a reduction in Treg-mediated suppression of T cell proliferation, means a decrease in the activity, proliferation, or development of suppressive immune cells after treatment compared with before treatment.

As used herein “suppression of” one or more of the suppressive immune cells defined herein, such as e.g., suppression of T cell proliferation, means to the reduce the activity, proliferation, or development of one or more of the suppressive immune cells defined herein.

EZH2 inhibitors described herein include e.g., small molecules or biologics that are capable of inhibiting EZH2 methyltransferase activity. Inhibition can be measured in vitro, in vivo, or from a combination thereof. In one aspect, the EZH2 inhibitors in the methods described herein are selected from EPZ-6438, EPZ005687, EPZ011989, EI1, GSK126, GSK343, UNC1999, as well as from those described in WO 2013/075083, WO 2013/075084, WO 2013/078320, WO 2013/120104, WO 2014/124418, WO 2014/151142, and WO 2015/023915. In one alternative aspect, the EZH2 inhibitors in the methods described herein are selected from

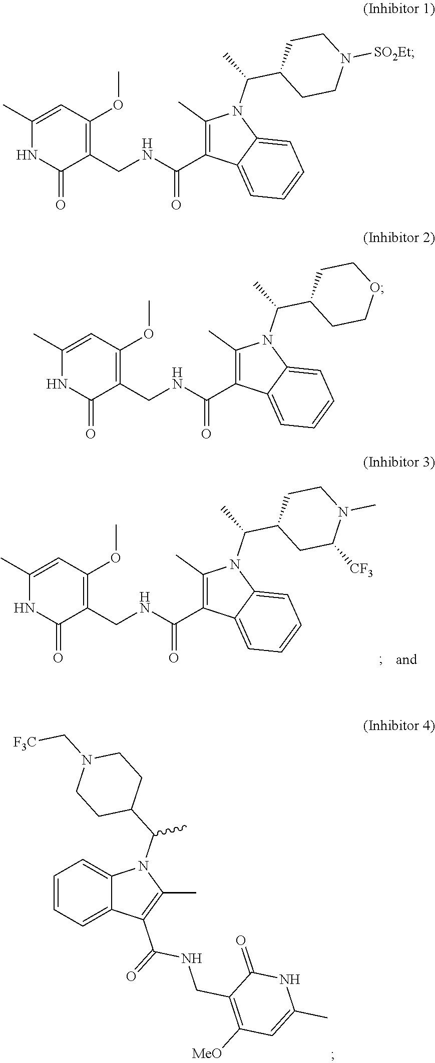

or a pharmaceutically acceptable salt thereof. In another alternative aspect, the EZH2 inhibitors in the methods described herein are

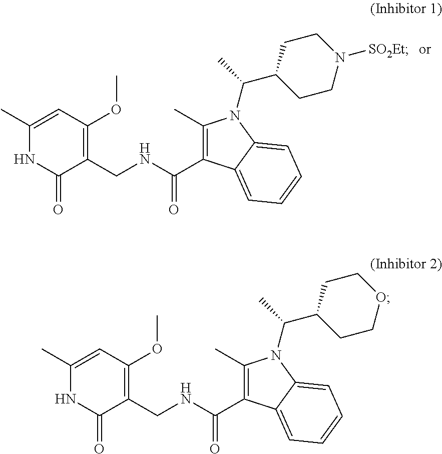

or a pharmaceutically acceptable salt thereof. In another alternative aspect, the EZH2 inhibitors in the methods described herein are

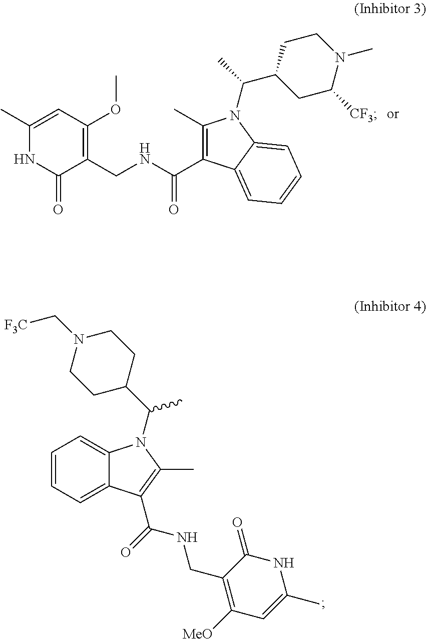

or a pharmaceutically acceptable salt thereof.

Also provided herein are methods of treating cancer in a subject in need thereof comprising administering to the subject an therapeutically effective amount of an EZH2 inhibitor as described herein and a therapeutically effective amount of a second agent that is an immunomodulator as defined herein.

The amount of an EZH2 inhibitor and an immunomodulator as defined herein is such that together, they elicit a synergistic effect to reduce the proliferation of regulatory T-cells (Tregs), increase cytotoxic T-cells (CD8), produce favorable CD8/Treg ratios, increase natural killer (NK) and natural killer T-cells (NKT), reduce M2 tumor-associate macrophages (TAMs), inhibit EZH2, and/or treat one or more cancers as described herein in a biological sample or in a patient.

Pharmaceutical compositions comprising an EZH2 inhibitor and an immunomodulator as described herein are also included.

As used herein, the terms “treatment,” “treat,” and “treating” refer to reversing, alleviating, or inhibiting the progress of a cancer, or one or more symptoms thereof, as described herein. Exemplary types of cancer include e.g., adrenal cancer, acinic cell carcinoma, acoustic neuroma, acral lentiginous melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogenous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligo astrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, primary peritoneal cancer, prostate cancer, pancreatic cancer, pharyngeal cancer, pseudomyxoma peritonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor.

In one aspect, the cancer treated by the methods or combinations described herein is selected from breast cancer, colorectal cancer, pancreatic cancer, cervical cancer, T cell lymphoma, uveal melanoma, gastric carcinoma, colorectal carcinoma, ovarian carcinoma, hepatocellular carcinoma, melanoma, and glioma. In another aspect, the cancer is selected from multiple myeloma, Hodgkin's lymphoma, non-Hodgkin's lymphoma, chronic lymphocytic leukemia, adult acute myeloid leukemia (AML), acute B lymphoblastic leukemia (B-ALL), and T-lineage acute lymphoblastic leukemia (T-ALL). In another aspect, the cancer is selected from multiple myeloma, Hodgkin's lymphoma, non-Hodgkin's lymphoma, chronic lymphocytic leukemia, adult acute myeloid leukemia (AML), squamous cell lung cancer, glioblastoma multiforme, and diffuse-type giant cell tumor. In another aspect, the cancer treated is non-Hodgkin's lymphoma.

Also included is the use of an EZH2 inhibitor as described herein in the manufacture of a medicament for the treatment of one or more cancers described herein, such as those cancers characterized by a high frequency of one or more suppressive immune cells. Also included herein are pharmaceutical compositions comprising an EZH2 inhibitor and an immunomodulator as described herein optionally together with a pharmaceutically acceptable carrier, in the manufacture of a medicament for the treatment of one or more cancers described herein, such as those cancers characterized by a high frequency of one or more suppressive immune cells. Also included is an EZH2 inhibitor for use in the treatment of a subject with cancer, such as those cancers characterized by a high frequency of one or more suppressive immune cells. Further included are pharmaceutical compositions comprising an EZH2 inhibitor and an immunomodulator as described herein, optionally together with a pharmaceutically acceptable carrier, for use in the treatment of one or more cancers described herein, such as those cancers characterized by a high frequency of one or more suppressive immune cells.

Further provided are packaged compositons comprising an effective amount of an EZH2 inhibitor described herein or a pharmaceutically acceptable salt thereof; and a pharmaceutically acceptable carrier or diluent, wherein the composition is packaged with instructions to treat a subject suffering from a cancer characterized by a high frequency of one or more suppressive immune cells. In one aspect, the packaged compositon further comprises an effective amount of an immumomodulator described herein.

The term “pharmaceutically acceptable carrier, adjuvant, or vehicle” refers to a non-toxic carrier, adjuvant, or vehicle that does not adversely affect the pharmacological activity of the compound with which it is formulated, and which is also safe for human use. Pharmaceutically acceptable carriers, adjuvants or vehicles that may be used in the compositions of this disclosure include, but are not limited to, ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., microcrystalline cellulose, hydroxypropyl methylcellulose, lactose monohydrate, sodium lauryl sulfate, and crosscarmellose sodium), polyethylene glycol, sodium carboxymethylcellulose, polyacrylates, waxes, polyethylene-polyoxypropylene-block polymers, polyethylene glycol and wool fat.

Compositions and method of administration herein may be orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Other forms of administration are as described in WO 2013/075083, WO 2013/075084, WO 2013/078320, WO 2013/120104, WO 2014/124418, WO 2014/151142, and WO 2015/023915, the contents of which are incorporated herein by reference.

### EXEMPLIFICATION

While have described a number of embodiments of this, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

Inhibitor 1 was prepared according to the procedures described in Bradley, W. D., et al. (2014). EZH2 Inhibitor Efficacy in Non-Hodgkin's Lymphoma Does Not Require Suppression of H3K27 Monomethylation. Chemistry & Biology 21, 1463-1475.

Inhibitor 4 was prepared according to the procedures described in WO 2013/120104.

Materials and Methods

Treg differentiation and RNA-seq. Leukopak samples were procured from the Biological Specialty Corporation (Colmar, Pa.) and peripheral blood mononuclear cells (PBMCs) were isolated by Ficoll (GE Biosciences) density gradient centrifugation. Naive CD4+ CD45RA+ T cells were isolated from PBMCs to a purity>98% using Miltenyi naive human T cell isolation kits (130-094-131, Miltenyi Biotech). Isolated cells were cultured at 10̂6 cells/mL under iTreg-polarizing conditions, using Human T-Activator CD3/CD28 Dynabeads® (11132D, Invitrogen), human TGFβ at 10 ng/mL and human IL-2 at 10 U/mL (100-B and 202-IL, respectively; R&D Biosystems). RNA was isolated from iTreg cultures at 6 h, 24 h, 3 d and 4 d post-activation using Qiagen RNeasy Plus mini kits and sequenced at Ocean Ridge Biosciences, FL. Reads from RNA-seq were mapped to the hg19 version of the human genome using TopHat v1.4.1 with parameters -p 2—library-type fr-unstranded. The hg19 bowtie genome index was downloaded from ftp://ftp.ccb.jhu.edu/pub/data/bowtie_indexes/. Duplicate read pairs were removed prior to further processing. Cufflinks was run against reference transcriptome Homo_sapiens.GRCh37.73.chr.gtf, obtained from ftp://ftp.ensembl.org/pub/release-73/gtf/homo_sapiens/Homo_sapiens.GRCh37.73.gtf.gz, with parameters—no-effective-length-correction and—library-type fr-unstranded. Failed expression estimate attempts were set to NA and ignored for the rest of the analyses.

ChIP. Naïve human CD4+ T cells were treated with 5 μM Inhibitor 1 or DMSO under iTreg polarizing conditions (described above) for 4 days. 4×107 cells were cross-linked in cell culture medium with 1% formaldehyde for 10 minutes. Formaldehyde-cross-linking was quenched using glycine at a final concentration of 125 mM for 10 minutes. Cells were pelleted and washed with PBS plus protease inhibitors. Cell pellets were flash frozen in liquid nitrogen and stored at −80° C. until ready to proceed. Cell pellets were thawed on ice with the addition of cold 1 ml of lysis buffer (1% SDS, 10 mM EDTA, 50 mM Tris-HCl, pH 8.1, protease inhibitors) and incubated on ice for 10 minutes. Sample was then sonicated on ice using a microtip probe sonicator (Branson) on setting 3.5 for 15 minutes total (cycle: 10 seconds on, 30 seconds off). Samples were clarified and supernatant was collected. Before adding antibodies to the lysate, additional ChIP dilution buffer (1.1% Triton X-100, 1.2 mM EDTA, 16.7 mM Tris-HCl, pH8.1, 167 mM NaCl) was added to lower the SDS concentration to 0.1%. For immunoprecipitation, 4 μg of anti-EZH2 antibody (07-689, Millipore) was added to chromatin from 2×107 cells and incubated at 4° C. overnight. For histone modification ChIPs, 4 μg of anti-H3K27me3 antibody (9733, Cell Signaling) was added to chromatin from 10×106 cells along with sonicated chromatin from 1.25×10⁵ Drosophila S2 cells and 2 μl anti-H2Av antibody (39715, Active Motif) used for normalization control and incubated at 4° C. overnight. Antibody-chromatin complexes were captured by addition of 50 μl protein G magnetic beads (Invitrogen) per sample. Bead-chromatin mixture was incubated with rotation for 1 hr at 4° C. Beads with bound antibody-protein-DNA were washed three times with RIPA wash buffer (0.1% SDS, 0.1% DOC, 1% Triton X 100, 1 mM EDTA, 10 mM Tris-HCl pH 8.1, 150 mM NaCl), three times with RIPA 500 wash buffer (0.1% SDS, 0.1% DOC, 1% Triton X 100, 1 mM EDTA, 10 mM Tris-HCl pH 8.1, 500 mM NaCl)), three times with LiCl wash buffer (0.5% DOC, 10 mM Tris-HCl pH 8.1, 250 mM LiCl, 0.5% Triton X-100)), and twice with TE (10 mM Tris-HCl pH 8.5, 1 mM EDTA). DNA-protein complexes were eluted from the beads with elution buffer (10 mM Tris-HCl pH 8, 10 mM EDTA, 0.1% SDS, 5 mM DTT) at 65° C. for 1 hour with intermittent agitation. Eluted chromatin underwent crosslink reversal at 65° C. for 4 hours. Uncrosslinked DNA was treated with 0.25 mg/ml RNase A at 37° C. for 30 minutes, followed by proteinase K digestion (0.25 mg/ml) for 1 hour at 55° C. DNA was purified by a PCR purification column (Qiagen MinElute), and eluted with buffer EB (10 mM Tris-HCl, pH 8).

Immunoprecipitated, purified DNA was used to generate uniquely barcoded libraries for each sample using the Ovation Ultralow DR Multiplex System (NuGEN), following the manufacturer's instructions. 10 ng of DNA was used for each library preparation. 17 cycles of PCR amplification were carried out for each library preparation. Three samples were multiplexed together using the different barcodes provided with the kit. After final bead purification step of library preparation, the DNA was run on a 2% agarose E-gel (Invitrogen) and DNA between 200-350 bp was extracted and purified via the Qiagen MinElute column. The quality of the DNA was assessed on an Agilent 2100 Bioanalyzer (Agilent Techologies) and then sequenced on an Illumina HiSeq at the BioMicro Center at MIT (Cambridge, Mass.).

Luminex cytokine assays. Cytokines were quantified from day 4 cell supernatants using Luminex multiplex assays (HTH17MAG-14K-12, Millipore), as per the manufacturer's protocol.

Treg suppression assay. Human iTregs were differentiated in vitro (as described above) for 4 days in the presence of DMSO or 5 μM Inhibitor 1. Cells were taken off Dynabead stimulation on day 4, washed and counted. Naïve T cells were labeled with CFSE (Carboxyfluorescein succinimidyl ester; C34554, Life Technologies) using the manufacturer's protocol. Cocultures of naïve T cells and iTregs were set up at ratios of 1:2, 1:4 and 1:8. Human T-Activator CD3/CD28 Dynabeads® were added at a 1:8 ratio of beads to cells.

Lentiviral shRNA knockdown of EZH2. Naïve human T cells were cultured under iTreg-inducing conditions, as described above and at ˜16 h post-activation were infected with lentivirus harboring shRNAs specific for EZH2 (3 independent hairpins per protein were cloned into pLKO.1-based lentiviral vectors; table below). Lentiviral supernatants were added to T cells in the presence of 8 μg/mL sequabrene (S2667-1VL, Sigma) followed by spin infection at 2100 rpm, 90′ at 30° C. Transduced cells were selected by addition of 1 μg/mL puromycin after 24 h; infection rates were monitored by measuring GFP fluorescence. Suppression assays were set up using day 7 iTreg cultures; naïve T cells were labeled with Cell Proliferation dye eFluor 450 (65-0842-90, eBioscience).

| shRNA | Target Sequence |
|---|---|
| EZH2 sh1 | GCTGAAGCCTCAATGTTTAGA |
| EZH2 sh2 | CGATCCTGAAGAAAGAGAAGA |
| EZH2 sh3 | GACTCTGAATGCAGTTGCTTC |

Tumor implantation and dosing. CT26 mouse colon carcinoma cells (ATCC CRL-2638) were expanded in vitro and 1×10⁵ cells/mouse were inoculated with 50% Matrigel into the subcutaneous flank region of 6-8 week old female BABL/C mice (Taconic). Mice were randomized once tumors were palpable (>200 mm³) and dosing was started on the same day. Inhibitor 1 was administered sub-cutaneously BID at 200 mg/kg and the PD-1 antibody (clone: RMP1-14, BE0146, BioXCell) was administered IP at 200 μg/mouse every 3-4 days. Tumors were measured and body weight recorded every 2-3 days.

Isolation and analysis of tumor-infiltrating cells. Tumors were chopped into small pieces, digested with 3 mg/mL collagenase A and 100 μg/mL DNase I at 37° C. for 30 mins, followed by the addition of FBS. The digested tumor mass was filtered using 40 μM filters, spun down and washed with PBS Immune cells were visualized and quantifed by FACS on the BD FACSCanto™ II flow cytometry analyzer (BD Bioscience).

Mouse antibodies. CD16/CD32 Purified (Fc block; 14-0161-82, eBioscience), CD45 eFluor® 450 (48-0451-82, eBioscience), CD3e PerCP-Cyanine5.5 (45-0031-82, eBioscience), CD4 APC (17-0041-82, eBioscience), CD8a PE-Cyanine7 (25-0081-82, eBioscience), CD45-FITC (11-0451-82, eBioscience), CD11b PE (12-0112-82, eBioscience), F4/80 Antigen PerCP Cyanine5.5 (45-4801-82, eBioscience), Ly-6C APC (17-5932-82, eBioscience), CD160 PE (12-1601-82, eBioscience), CD335 (NKp46) PE-Cyanine7 (25-3351-82, eBioscience), NK1.1 APC-eFluor® 780 (47-5941-82, eBioscience), IFNγ APC (17-7311-82, eBioscience), TNFα PE (12-7321-82, eBioscience), CD3ϵ APC-eFluor® 780 (47-0031-82, eBioscience), CD19 APC-eFluor® 780 (47-0193-82, eBioscience), Ki-67 FITC (11-5698-82, eBioscience), CD4 APC-eFluor® 780 (47-0041-82, eBioscience), MHC-II (M/114.15.2)-AmCyam/V500 (562366, BD Biosciences), CD8α AmCyan/V500 (560778, BD Biosciences), CD206 FITC (MRD5D3) (MCA2235FA, AbD Serotec), Ly-6G PE/Cy7 (127617, Biolegend).

PRC2 Core Components are Up-Regulated During Human Treg Differentiation and its Catalytic Component, EZH2, is an Essential Modulator of Chromatin Structure in these Cells.

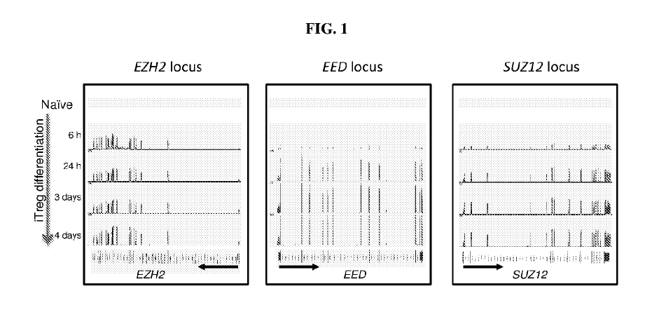

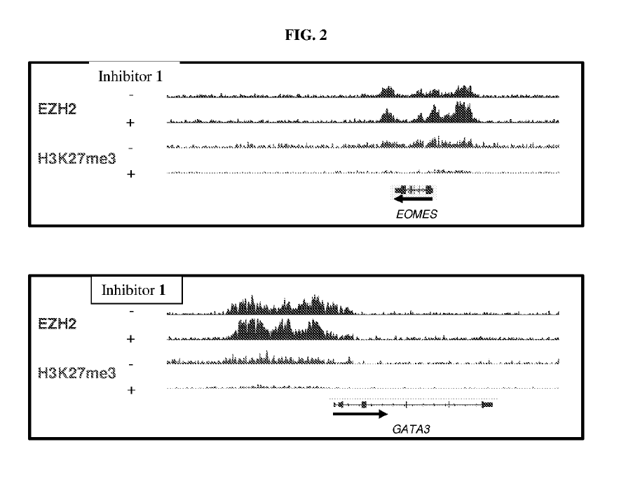

In order to explore the role of EZH2 in Treg biology, we started by investigating if EZH2, along with PRC2 core components EED and SUZ12, was expressed during Treg differentiation. With that aim we performed RNA sequencing (RNA-seq) from naïve human T cells and T cells differentiating along the Treg lineage pathway (naïve T cells activated through the T cell receptor in the presence of TGF-β1 and IL-2) at 6 hours, 24 hours, 3 days and 4 days. The expression levels of EZH2, EED and SUZ12 are all below detection in naïve T cells. However, during differentiation into Tregs, all 3 PRC2 components are induced as early as 6 h, and remain highly expressed for the remainder of the differentiation period studied (4 days; FIG. 1). These observations are consistent with the notion of PRC2 playing a role human Treg differentiation. Because EZH2, the catalytic component of PRC2, drives tri-methylation of lysine 27 in histone H3 (H3K27me3) we used a potent and selective EZH2 small molecule inhibitor, Inhibitor 1, to investigate if in fact EZH2 was not only expressed, but biochemically active in human Treg cells. Indeed, chromatin immuno-precipitation followed by deep sequencing (ChIP-seq) demonstrates that EZH2 binds to certain repressed loci in these cells, and its inhibition results in loss of H3K27 tri-methylation (FIG. 2).

EZH2 is Not Necessary for FOXP3 Expression.

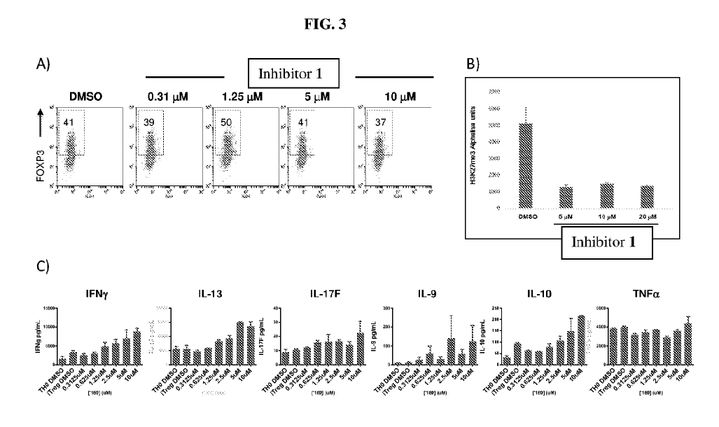

The transcription factor FOXP3 is known to be an essential regulator of Treg cells, and therefore is important to understand if EZH2 plays any role in its expression. With that purpose, we differentiated naïve human T cells into Tregs in the presence of increasing concentrations of Inhibitor 1, and analyzed the cultures by flow cytometry (FACS). EZH2 inhibition had no impact in FOXP3 expression, as we detected no differences in the frequencies of FOXP3⁺ cells between the Inhibitor 1-treated cultures and those treated with DMSO control (FIG. 3A). This lack of effect in FOXP3 expression is not attributable to lack of biochemical activity of the compound, as H3K27me3 is robustly decreased in the same cells (FIG. 3B). Moreover, we observed a dose-dependent increase in the expression of certain genes, as seen by secretion to the culture medium of the encoded proteins, IFNγ, IL-13 and IL-10 (FIG. 3C). Taken together, these observations suggest that although EZH2 is dispensable for FOXP3 expression, it might play an important role in the function of human Treg cells.

EZH2 is Functionally Required for Human Treg Cell Activity.

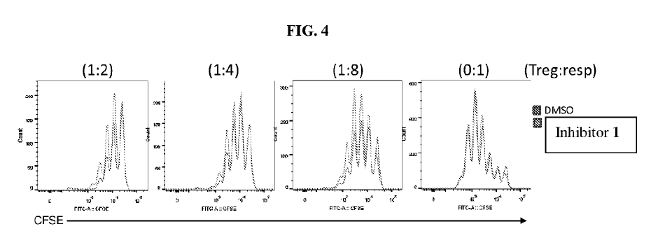

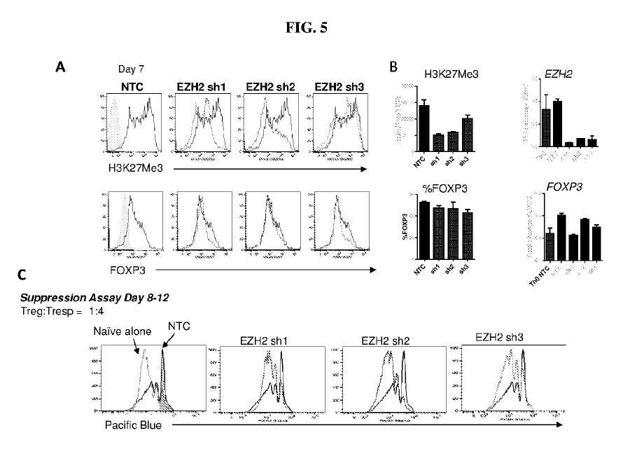

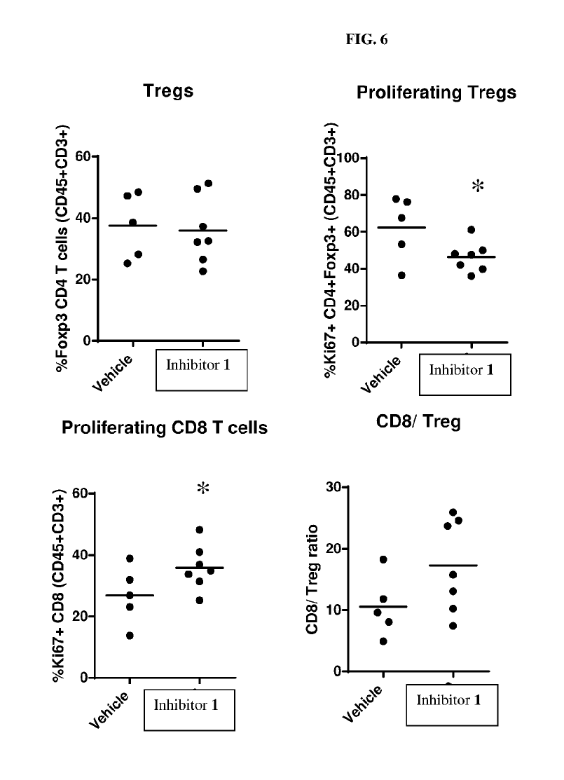

A fundamental biological function of Treg cells is the suppression of proliferation of other immune cells, including T cells. These functions can be investigated in vitro in so-called suppression assays, where Treg cells are co-cultured with naïve T cells (“responder cells”, Tresp), whose proliferation can be followed by progressive dilution of the FACS dyes CSFE or Pac-Blue. In order to explore any functional consequence of EZH2 inhibition, we performed suppression assays using Treg cells that had been differentiated in the presence of Inhibitor 1 or DMSO control. As expected, increasing ratios of DMSO-treated Treg cells to Tresp cells resulted in increased suppression of Tresp proliferation. However, Treg cells differentiated in the presence of Inhibitor 1 were impaired in their suppressive capability (FIG. 4), demonstrating that EZH2 catalytic activity is required for full biological activity of Treg cells. Importantly, Inhibitor 1 had no impact on proliferation of Tresp alone (FIG. 4). To rule out any possibility of an off-target effect of Inhibitor 1, and to confirm the functional requirement of EZH2 for Treg activity with an orthogonal assay, EZH2 expression was reduced by lentiviral transduction of 3 independent EZH2 shRNA hairpins in human T cells under Treg cell differentiating conditions. Cells transduced with shRNAs targeting EZH2, but not the non-targeting control (NTC), showed significantly reduced EZH2 mRNA levels and reduced global H3K27me3 levels, with minimal impact on FOXP3 protein expression (FIG. 5). In suppression assays, all 3 hairpins reduced the suppressive capacity of Treg cells (FIG. 5). Together, these data demonstrate that EZH2 is functionally required for the suppressive capability of human Treg cells, and that this function depends on its catalytic activity.

EZH2 Inhibition Results in Tumor Growth Inhibition in the CT26 Allograft Transplantation Tumor Model.

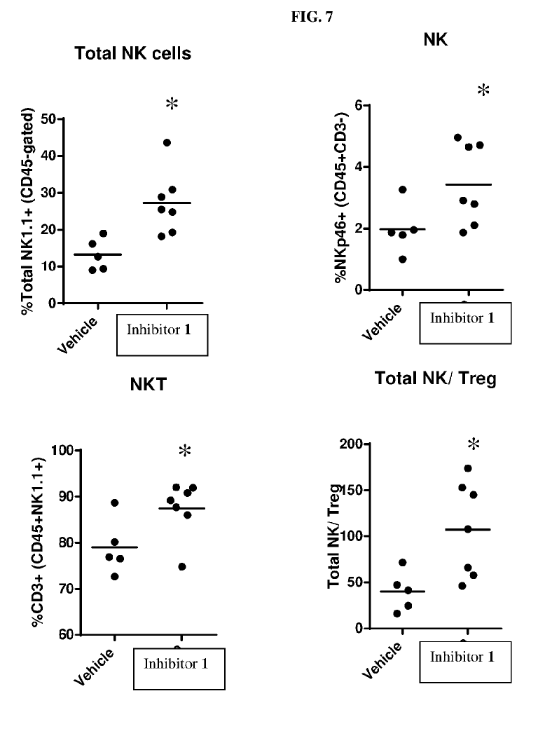

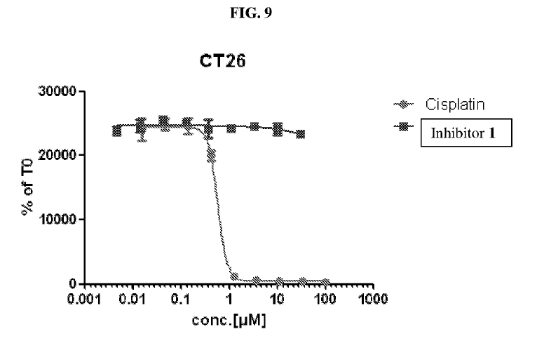

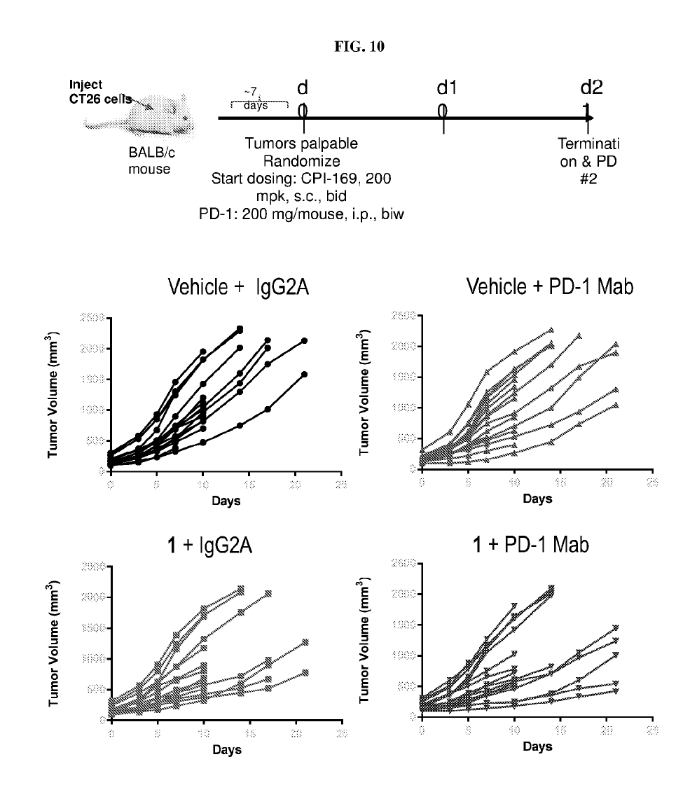

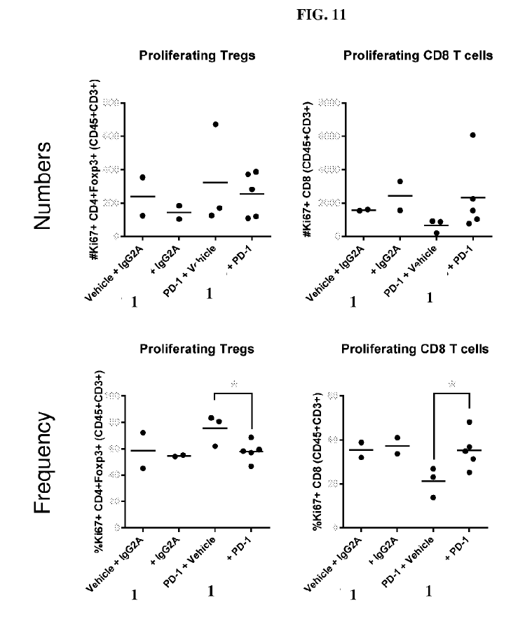

We first evaluated whether treatment treatment of CT26 cancer cells would be sensitive to EZH2 ihibition. Protocols using CT26 cancer cells as a model for testing immunotherapy protocols and in studies on host immune response are known. Despite the favorable results on modulating Tregs, CD8 cells, NK and NKT cells, and M2 macrophages (FIG. 6-8), it was disconcerting to find substantially no CT26 cell sensitivity with administration of Inhibitor 1 (FIG. 9). Regardless, based on our discovery of a functional requirement of EZH2 for Treg cell suppressive capability, and because suppressive pathways are known to be co-opted by tumor cells in vivo to evade immune attack, we tested Inhibitor 1 in an in vivo efficacy study. We inoculated BALB/c mice with syngeneic CT26 cancer cells (insensitive to EZH2 inhibition, Inhibitor 1) and once tumors were palpable animals were randomized into 4 treatment groups: Vehicle control; anti-PD1 antibody (PD-1); Inhibitor 1; combination PD1+Inhibitor 1. The PD1 treatment group showed only marginal efficacy after the 21-day treatment period. However, treatment with Inhibitor 1 was significantly efficacious. The combination group was also efficacious, with a trend towards increased efficacy compared to all other groups (FIG. 10).

EZH2 Inhibition Alters the Tumor Immune Response In Vivo.

The observed efficacy pictured in FIG. 11 was accompanied by changes in the immune infiltrate present in the tumors. Tumors were isolated from mice upon termination of the study (day 22), digested and stained for immune markers allowing for quantification of immune cell populations by FACS. The Inhibitor 1+αPD1 group exhibited significant reduction in the proportion of proliferating Tregs and significant increase in proliferating CD8 T cells, relative to the PD1 group alone; *p≤0.05, Student's T-test. Consistent with the reduced efficacy observed in the αPD1 group, we found increased proliferating Treg cells in this group along with decreased proliferating cytotoxic CD8 cells, especially when compared to the combination group (FIG. 11). This effect was also clear if groups treated with the EZH2 inhibitor were analyzed together, and against groups not treated with the EZH2 inhibitor (FIG. 6).

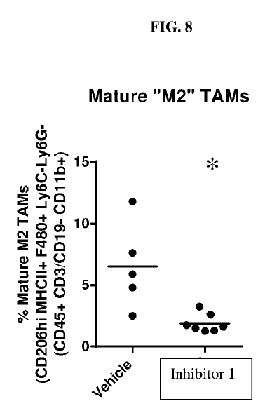

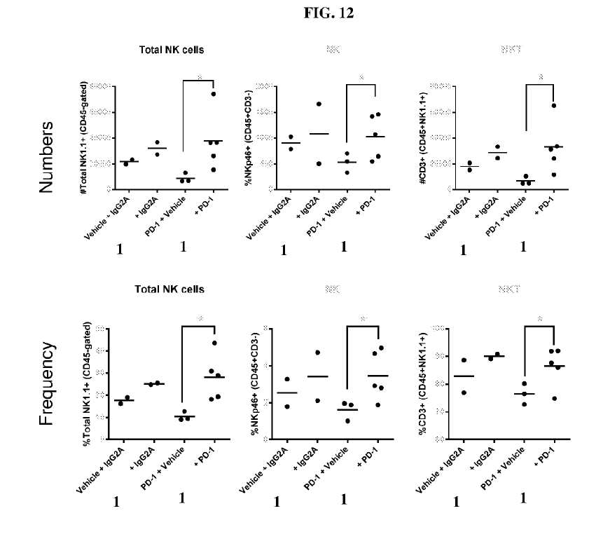

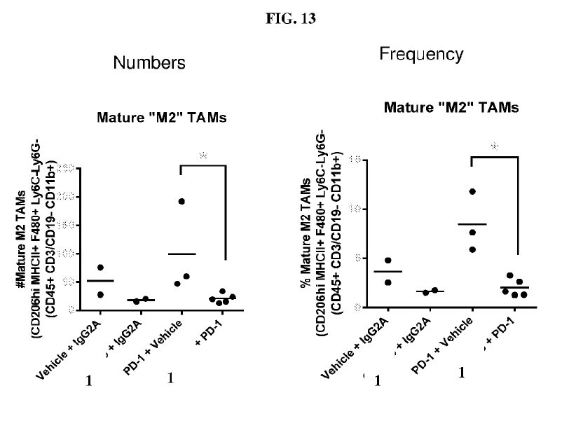

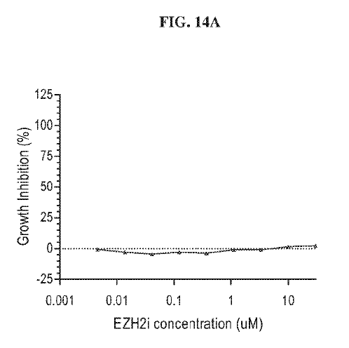

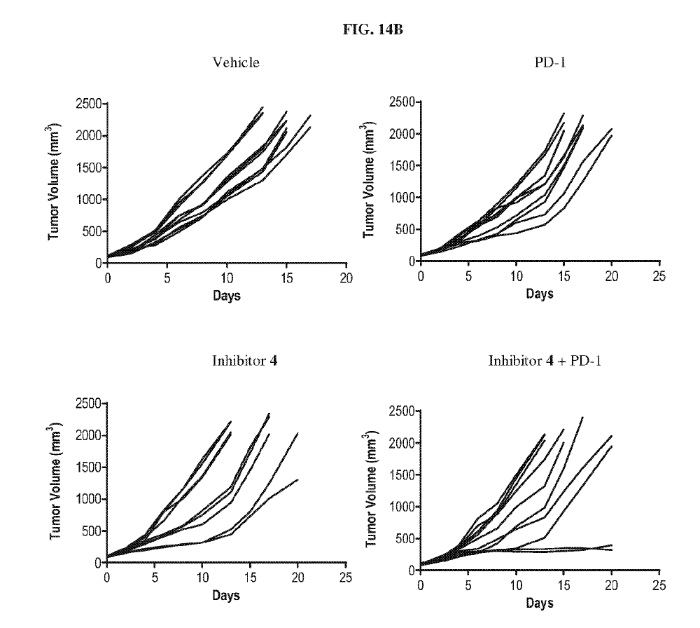

CD8 T cells are known to be major effectors of anti-tumor efficacy in mice and humans. We also explored immune cell changes beyond what our in vitro experiments had predicted, and found several unanticipated observations. First, natural killer (NK) and NKT cells, also major drivers of anti-tumor activity were increased in Inhibitor 1-treated animals, while reduced in the αPD1 group (FIG. 12). This effect was also clear if groups treated with the EZH2 inhibitor were analyzed together, and against groups not treated with the EZH2 inhibitor (FIG. 7). Second, M2 tumor associated macrophages (TAM), known to be immuno-suppressive, reduced in Inhibitor 1-treated animals, while increased in αPD1-treated animals (FIG. 13). This effect was also observed if groups treated with the EZH2 inhibitor were analyzed together, and against groups not treated with the EZH2 inhibitor (FIG. 8). A reduction of tumor volume for treatment with Inhbitor 1+αPD1 is shown in FIG. 10. Similar results were also seen with Inhibitor 4. Growth of CT26 mouse colon carcinoma cells was assessed in the presence of increasing amounts of Inhibitor 4 concentrations. Inhibitor 4. As shown by FIG. 14A, Inhibitor 4 did not cause a significant viability defect compared to CT26 cells that were grown in the absence of Inhibitor 4. However, when the effect of Inhibitor 4 as a single agent or in combination with Anti-mouse PD1 antibodies was assessed in immune-competent Balb/c mice inoculated with CT26 cells, Inhibitor 4 in combination with Anti-PD1 showed complete abrogation of tumor growth in a subset of animals (FIG. 14B). In the absence Anti-PD1, Inhibitor 4 showed modest delay in tumor growth compared to the vehicle treated control animals

Taken together, these novel data demonstrate that EZH2 inhibition is a viable approach in cancer immunotherapy, as a single agent and in combination with checkpoint inhibitors, such as anti-PD1 antibodies.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A method of treating a subject with a cancer characterized by a high frequency of one or more suppressive immune cells, comprising administering to the subject a therapeutically effective amount of an enhancer of zeste homolog 2 (EZH2) inhibitor.

2. The method of claim 1, wherein prior to treatment, the cancer was determined to comprise a high frequency of one or more suppressive immune cells.

3. The method of claim 1, comprising the step of performing a biopsy of the subject's cancer prior to treatment and determining if the cancer comprises a high frequency of one or more suppressive immune cells.

4. A method of treating a subject with a cancer comprising determining the frequency of one or more suppressive immune cells in the cancer; and administering to the subject a therapeutically effective amount of an EZH2 inhibitor, if the subject's cancer comprises a high frequency of one or more suppressive immune cells.

5. (canceled)

6. The method of claim 4, further comprising administering to the subject a therapeutically effective amount of a cancer therapy other than the administration of an EZH2 inhibitor, if the frequency of one or more suppressive immune cells of the subject's cancer is not high.

7. The method of claim 1, further comprising administering a therapeutically effective amount of an immunomodulator.

8. The method of claim 7, wherein the EZH2 inhibitor is administered concurrently with the immunomodulator.

9. The method of claim 8, wherein the immunomodulator is selected from immune checkpoint blockade inhibitors, cell based therapies, vaccination strategies, agents that prevent metabolic inhibition of immune responses, and cytokine-based therapies.

10. The method of claim 9, wherein the immunomodulator is an immune checkpoint blockade inhibitor.

11. The method of claim 10, wherein the immunomodulator is an immune checkpoint blockade inhibitor selected from anti-CTLA4, ipilimumab, nivolumab, pembrolizumab, pidilizumab, BMS 936559, atezolizumab, avelumab, anti-CD47, PD-1 antibody, anti-PDL1, lambrolizumab, AMP-224, and MEDI-4736.

12. The method of claim 11, wherein the immunomodulator is an immune checkpoint blockade inhibitor selected from anti-CTLA4, ipilimumab, nivolumab, pembrolizumab, pidilizumab, BMS 936559, atezolizumab, anti-CD47, PD-1 antibody, anti-PDL1, lambrolizumab, AMP-224, and MEDI-4736.

13. The method of claim 12, wherein the immunomodulator is αPD-1 antibody.

14. The method of claim 1, wherein the cancer is characterized by a high frequency of regulatory T cells, a high frequency of M2 tumor associated macrophages, or both a high frequency of regulatory T cells and a high frequency of M2 tumor associated macrophages.

15. The method of claim 14, wherein the cancer is characterized by a high frequency of regulatory T cells defined by a median cut-off value of 1.5, 2, 2.5, 3, 3.5, 4, 4.5, 5, 5.5, 6, or 6.5 intratumor Tregs in a tissue sample taken from the cancer per high-powered microscopic field, a high frequency of M2 tumor associated macrophages defined by a median cut-off value of 1.5, 2, 2.5, 3, 3.5, 4, 4.5, 5, 5.5, 6, or 6.5 intratumor M2 tumor associated macrophages in a tissue sample taken from the cancer per high-powered microscopic field or both a high frequency of regulatory T cells and a high frequency of M2 tumor associated macrophages defined by a median cut-off value of 1.5, 2, 2.5, 3, 3.5, 4, 4.5, 5, 5.5, 6, or 6.5 intratumor Tregs in a tissue sample taken from the cancer per high-powered microscopic field and a median cut-off value of about 1.5, 2, 2.5, 3, 3.5, 4, 4.5, 5, 5.5, 6, or 6.5 intratumor M2 tumor associated macrophages in a tissue sample taken from the cancer per high-powered microscopic field.

16. The method of claim 1, wherein the cancer is selected from breast cancer, colorectal cancer, pancreatic cancer, cervical cancer, T cell lymphoma, uveal melanoma, gastric carcinoma, colorectal carcinoma, ovarian carcinoma, hepatocellular carcinoma, melanoma, and glioma.

17. The method of claim 1, wherein the cancer is selected from multiple myeloma, Hodgkin's lymphoma, non-Hodgkin's lymphoma, chronic lymphocytic leukemia, adult acute myeloid leukemia (AML), squamous cell lung cancer, glioblastoma multiforme, and diffuse-type giant cell tumor.

18. The method of claim 1, wherein the cancer is Hodgkin's lymphoma.

19. A method of treating a subject with a cancer comprising administering a therapeutically effective amount of an EZH2 inhibitor; determining if after administration of the EZH2 inhibitor a reduction in Treg-mediated suppression of T cell proliferation occurred, a suppression of M2 tumor-associated macrophages occurred, or an increase in the frequency of natural killer cells (NK) cells occurred, or a combination thereof; and continuing to administer a therapeutically effective amount of an EZH2 inhibitor if there has been a reduction in Treg-mediated suppression of T cell proliferation, a suppression of tumor-associated macrophages, or an increase in the frequency of natural killer cells (NK) cells, or a combination thereof.

20. A method of treating a subject with a cancer comprising administering a therapeutically effective amount of an EZH2 inhibitor; determining if after administration of the EZH2 inhibitor a reduction in Treg-mediated suppression of T cell proliferation occurred, a suppression of M2 tumor-associated macrophages occurred, or an increase in the frequency of natural killer cells (NK) cells occurred, or a combination thereof; and administering to the subject a therapeutically effective amount of a cancer therapy other than the administration of an EZH2 inhibitor if a reduction in Treg-mediated suppression of T cell proliferation did not occur, a suppression of tumor-associated macrophages did not occur, and an increase in the frequency of natural killer cells (NK) cells did not occur; and continuing to administer a therapeutically effective amount of an EZH2 inhibitor if there has been a reduction in Treg-mediated suppression of T cell proliferation, a suppression of M2 tumor-associated macrophages, or an increase in the frequency of natural killer cells (NK) cells, or a combination thereof.

21. The method of claim 1, wherein the EZH2 inhibitor is selected from EPZ-6438, 3-deazaneplanocin A (DZNep), EPZ005687, EPZ011989, EI1, GSK126, GSK343, UNC1999, EPZ-6438.

22. The method of claim 1, wherein the EZH2 inhibitor is 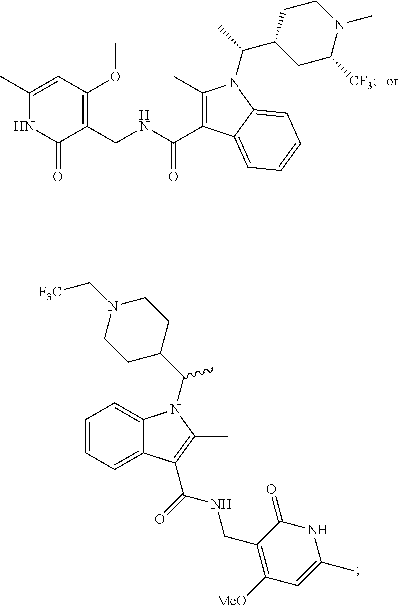
or a pharmaceutically acceptable salt thereof.

23. (canceled)

24. (canceled)


SEQUENCE LISTING (3 sequences)

SEQ ID NO: 1 - DNA, 21 bp, Artificial Sequence
  source: /note=“Description of Artificial Sequence Synthetic oligonucleotide”
gctgaagcctcaatgtttaga

SEQ ID NO: 2 - DNA, 21 bp, Artificial Sequence
  source: /note=“Description of Artificial Sequence Synthetic oligonucleotide”
cgatcctgaagaaagagaaga

SEQ ID NO: 3 - DNA, 21 bp, Artificial Sequence
  source: /note=“Description of Artificial Sequence Synthetic oligonucleotide”
gactctgaatgcagttgcttc